(19) United States
(12) Patent Application Publication
(10) Pub. No.: US 2026/0176584 A1
(43) Pub. Date: Jun. 25, 2026

(54) MACROPHAGE-MODIFICATION TECHNIQUE FOR TREATING METASTATIC SOLID TUMORS

(71) Applicant: Florida State University Research Foundation, Inc., Tallahassee, FL (US)

(72) Inventor: Jingjiao Guan, Tallahassee, FL (US)

(21) Appl. No.: 19/376,439

(22) Filed: Oct. 31, 2025

Related U.S. Application Data
(60) Provisional application No. 63/736,247, filed on Dec. 19, 2024.

(51) Int. Cl. C12N 5/0786; A61K 35/15; A61K 40/17; C12N 15/11

(52) U.S. Cl. CPC C12N 5/0645; A61K 35/15; A61K 40/17; C12N 15/11; C12N 2500/40; C12N 2500/50; C12N 2523/00

(57) ABSTRACT

Systems and methods are provided that can keep adoptively transferred macrophages in solid tumors in the tumoricidal MI phenotype. Microparticles containing poly(N-isopropylacrylamide) (PNIPAM) and a multilayered structure made of deoxyribonucleic acid (DNA) and a polycation (PC) can be produced by depositing a multilayer of DNA and a PC and a layer of PNIPAM on a stamp bearing micropillars, followed by printing the disk-shaped structures on a substrate. The DNA/PC multilayer can be delivered into the cytoplasm of the macrophages and/or monocytes by incubating the microparticles with macrophages and/or monocytes, briefly exposing the cells to a cold temperature, and continuing to culture the cells. The microparticles can be phagocytosed by the macrophages and/or monocytes during initial incubation. The brief exposure to the cold temperature can induce phagosomal rupture and release of the DNA/PC multilayer into the cytoplasm of the cells.

### CROSS-REFERENCE TO RELATED APPLICATION

This application claims the benefit of U.S. Provisional Application Ser. No. 63/736,247, filed Dec. 19, 2024, the disclosure of which is hereby incorporated by reference in its entirety, including all figures, tables, and drawings.

### BACKGROUND

Metastatic solid tumors are responsible for the majority of cancer deaths. Chimeric antigen receptor (CAR) T-cell therapy is highly effective for treating hematologic cancers, but ineffective for treating metastatic solid tumors partially due to the inability of the CAR T-cells to penetrate into the solid tumors. On the contrary, circulating monocytes can accumulate in metastatic solid tumors and then differentiate into macrophages known as tumor-associated macrophages (TAMs). However, the TAMs are typically polarized by the immunosuppressive tumor microenvironment to an M2 phenotype, which promotes tumor growth and metastasis. This fact inspired an anticancer therapy, adoptive macrophage therapy (AMT), based on delivering macrophages with an M1 phenotype, which is tumoricidal, into the tumors. AMT includes extracting monocytes from cancer patients, expanding and polarizing them into M1 macrophages, and re-infusing them back into the patients. The early version of AMT was proven ineffective by clinical trials conducted during 1987-2010. The past decade has seen continuous efforts to develop novel AMT techniques, but these techniques are either at the stage of animal studies or in phase 1 clinical trials.

### BRIEF SUMMARY

Embodiments of the subject invention provide novel and advantageous systems and methods that can keep adoptively transferred macrophages in solid tumors in the tumoricidal M1 phenotype. These systems and methods can significantly improve adoptive macrophage therapy (AMT) towards a curative therapy for metastatic solid tumors. Microparticles containing poly(N-isopropylacrylamide) (PNIPAM) and a multilayered structure made of deoxyribonucleic acid (DNA) and a polycation (PC) can be produced by depositing a multilayer of DNA and a PC and a layer of PNIPAM on a stamp bearing micropillars, followed by printing the disk-shaped structures on a substrate. The substrate can be coated, such as with a thin layer of material. The DNA/PC multilayer can be delivered into the cytoplasm of the macrophages and/or monocytes by incubating the microparticles with macrophages and/or monocytes, briefly exposing the cells to a cold temperature, and continuing to culture the cells. The microparticles can be phagocytosed by the macrophages and/or monocytes during initial incubation. The brief exposure to the cold temperature can induce phagosomal rupture and release of the DNA/PC multilayer into the cytoplasm of the cells. The DNA in the DNA/polycation multilayer can promote the cells towards a proinflammatory state, known as M1 state, and maintain it for an extended period.

In an embodiment, a method for maintaining an immune cell in a solid tumor in a tumoricidal M1 phenotype can comprise: a) incubating the immune cell with a microdisk comprising a layer of a first polymer (e.g., PNIPAM) and a multilayer of DNA and a PC, the incubating being performed at a first temperature (e.g., at least 30° C.) for a first period of time; b) after step a), exposing the immune cell and the microdisk to a second temperature (e.g., less than 10° C.) for a second period of time; and c) after step b), culturing the immune cell at a third temperature (e.g., at least 30° C.) for a third period of time. The first temperature can be, for example, 37° C. or about 37° C. The second temperature can be, for example, 0° C. or about 0° C. The third temperature can be, for example, 37° C. or about 37° C. Step a) can cause the microdisk to be phagocytosed by the immune cell. Step b) can cause phagosomal rupture and release of the multilayer of DNA and PC into a cytoplasm of the immune cell. Step b) can cause the first polymer (e.g., PNIPAM) to dissolve. The first period of time can be in a range of, for example, from 0.5 hours to 30 hours (e.g., 2 hours or about 2 hours); the second period of time can be in a range of, for example, from 0.5 minutes to 10 minutes (e.g., 2 minutes or about 2 minutes); and/or the third period of time can be in a range of, for example, from 10 hours to 48 hours (e.g., 24 hours or about 24 hours). The microdisk can have a thickness in a range of, for example, from 0.1 micrometers (μm) to 3 μm (e.g., 1 μm or about 1 μm) and a diameter in a range of, for example, from 2 μm to 10 μm (e.g., 5 μm or about 5 μm). The method can further comprise, before step a): i) depositing the multilayer of DNA and PC on a stamp comprising pillars; ii) depositing the layer of first polymer (e.g., PNIPAM) on the multilayer of DNA and PC; and iii) printing the microdisk on a substrate by disposing the stamp comprising the layer of first polymer and the multilayer of DNA and PC on the substrate. The method can further comprise, before step iii), coating the substrate with a second polymer (e.g., polyvinyl alcohol (PVA)). The immune cell can be a macrophage (e.g., an adoptively transferred macrophage) or a monocyte.

In another embodiment, a method of treating a disease in a patient (e.g., a mammalian patient, such as a human patient) can comprise: isolating immune cells from the patient; modifying the immune cells ex vivo by applying the method described in the previous paragraph to the immune cells (including any or all features from the previous paragraph); and re-infusing the modified immune cells back to the patient.

### BRIEF DESCRIPTION OF DRAWINGS

FIG. 1 shows a step-by-step view of production of microparticles.

FIG. 2 shows a step-by-step view of delivery of a DNA/polycation multilayer into a cytoplasm of macrophages and/or monocytes to promote the cells to M1 state. It can start with allowing a (polycation/DNA)ₙ/PNIPAM microparticle to be phagocytosed into a phagosome of a macrophage, bursting the phagosome by exposing the macrophage to a temperature below 10° C. (e.g., 0° C. or about 0° C.) for a period of time (e.g., 0.5-10 minutes, such as 2 minutes or about 2 minutes) to release the dissolved PNIPAM and (PC/DNA), microdisk into the cytosol of the macrophage. Here, PNIPAM represents poly(N-isopropylacrylamide) and PC represents polycation. Next, the dissolved PNIPAM can be condensed into droplets (e.g., at a temperature of at least 25° C., such as 37° C. or about 37° C.) for a period of time (e.g., 10 hours-48 hours, such as 24 hours or about 24 hours). Then, the macrophage can be allowed to be polarized into M1 phenotype by the DNA in the (PC/DNA), microdisk.

FIG. 3 shows a view of general utility of embodiments of the subject invention.

FIG. 4A shows a fluorescence image. The scale bar is 50 micrometers (μm).

FIG. 4B shows a merged bright-field (gray) and fluorescence (red) image for a temperature of 37° C.

FIG. 4C shows a merged bright-field (gray) and fluorescence (red) image for a temperature of more than 22° C. and less than 37° C. The scale bar is 20 μm.

FIG. 4D shows a merged bright-field (gray) and fluorescence (red) image for a temperature of about 0° C. The scale bar is 20 μm.

FIG. 4E shows a merged bright-field (gray) and nanoparticle fluorescence (red) for a temperature of 37° C. The scale bar is 20 μm.

FIG. 4F shows a merged bright-field (gray) and nanoparticle fluorescence (red) and calcein AM (green) image for a temperature of 37° C. The scale bar is 20 μm.

FIG. 4G shows a merged bright-field (gray) and nanoparticle fluorescence (red) image and YOYO-1 fluorescence (green) image.

FIG. 4H shows a merged bright-field (gray) and nanoparticle fluorescence (red) image and YOYO-1 fluorescence (green) image for a temperature of about 0° C. The scale bar is 20 μm.

FIG. 4I shows a merged bright-field (gray) and nanoparticle fluorescence (red) image and YOYO-1 fluorescence (green) image. The scale bar is 5 μm.

FIG. 4J shows a merged bright-field (gray) and nanoparticle fluorescence (red) image and YOYO-1 fluorescence (green) image for a temperature of about 37° C. The scale bar is 20 μm.

### DETAILED DESCRIPTION

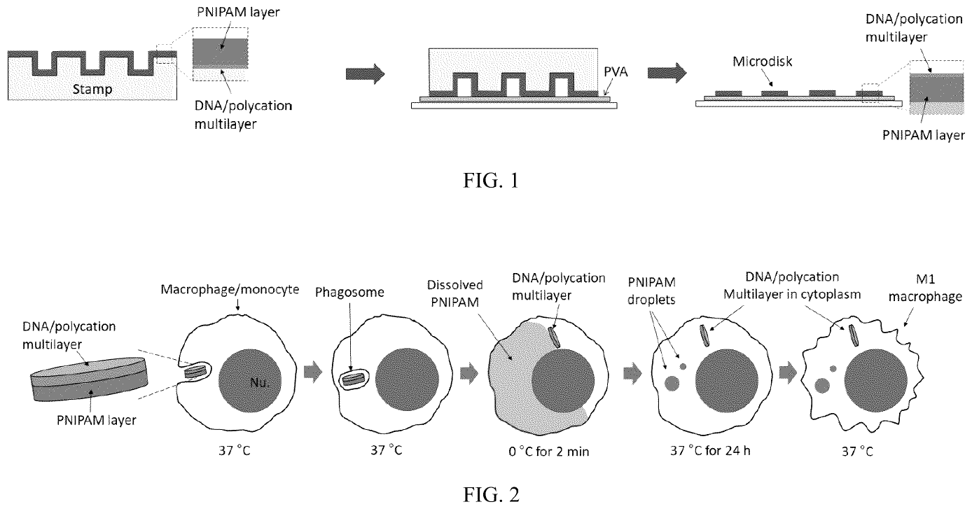

Embodiments of the subject invention provide novel and advantageous systems and methods that can keep adoptively transferred macrophages in solid tumors in the tumoricidal M1 phenotype. These systems and methods can significantly improve adoptive macrophage therapy (AMT) towards a curative therapy for metastatic solid tumors. Microparticles containing poly(N-isopropylacrylamide) (PNIPAM) and a multilayered structure made of deoxyribonucleic acid (DNA) and a polycation (PC) can be produced by depositing a multilayer of DNA and a PC and a layer of PNIPAM on a stamp bearing micropillars, followed by printing the disk-shaped structures on a substrate. The substrate can be coated, such as with a thin layer (e.g., thickness of less than 10 micrometers (μm), such as less than 1 μm) of material (e.g., polyvinyl alcohol (PVA)) (see also FIG. 1). The DNA/PC multilayer can be delivered into the cytoplasm of the macrophages and/or monocytes by incubating the microparticles with macrophages and/or monocytes, briefly exposing the cells to a cold temperature (such as below 10° C.; e.g., 0° C. or about 0° C.), and continuing to culture the cells (such as at a temperature of at least 25° C.; e.g., 37° C. or about 37° C.) (see also FIG. 2). The microparticles can be phagocytosed by the macrophages and/or monocytes during initial incubation. The brief exposure to the cold temperature can induce phagosomal rupture and release of the DNA/PC multilayer into the cytoplasm of the cells. The DNA in the DNA/polycation multilayer can promote the cells towards a proinflammatory state, known as M1 state, and maintain it for an extended period (e.g., at least 24 hours).

Metastatic solid tumors are a major cause of cancer mortality. The effectiveness of AMT, which harnesses macrophages' natural tumor-targeting abilities and their potential for tumoricidal M1 polarization, is hindered by the tumor microenvironment that typically induces a tumor-promoting M2 phenotype in these cells. Embodiments of the subject invention can maintain adoptively transferred macrophages in their M1 tumoricidal state within solid tumors. An important innovation is production of specially engineered microparticles for delivering double-stranded DNA, formulated into micrometer-sized DNA-PC complexes (which can be referred to as DNA-polycation microdisks) directly into the cytosol of macrophages. The presence of these DNA-polycation microdisks in the cytosol can effectively polarize and sustain macrophages in the M1 phenotype even within an M2-promoting tumor microenvironment.

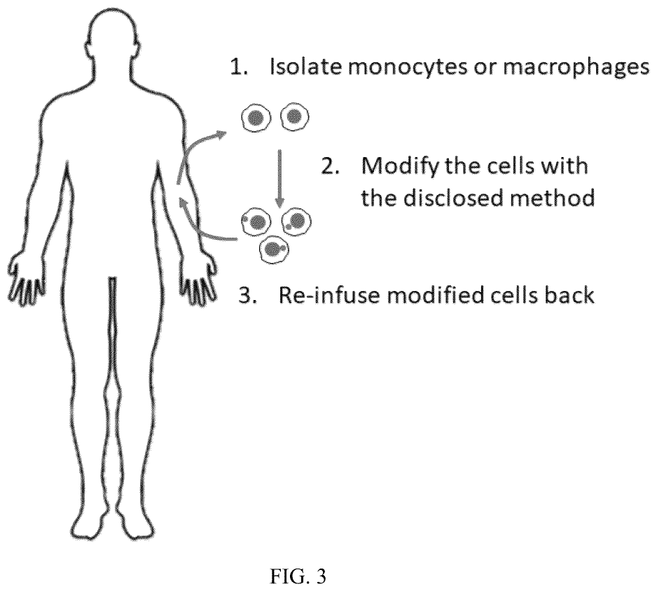

Embodiments of the subject invention can be used in, for example, AMT for treating human diseases, as schematically illustrated in FIG. 3. The therapy can include: isolating monocytes and/or macrophages from a patient; modifying the cells ex vivo with a system/method of an embodiment of the subject invention; and re-infusing the cells back to the patient.

One existing M1-polarization method intended to be used in AMT is based on treating macrophages with interferon γ (IFNγ) ex vivo [7]. Given that the tumor microenvironment can polarize TAMs to M2 phenotype, lack of an ability to continuously promote M1 polarization is a major shortcoming of this method. Another method attaches IFNγ-releasing microparticles to the external surface of therapeutic macrophages [9]. However, it is concerned with whether the surface-attached microparticles would reduce the ability of the systemically administered macrophages to accumulate in the tumors in humans. Another method relies on feeding iron oxide nanoparticles (IONPs) to macrophages ex vivo [10]. However, the M1-polarization mechanism of this method is restricted by the use of IONPs. Whether this mechanism is effective for treating human solid tumors is unknown. Another method is based on genetic modification of macrophages into chimeric antigen receptor (CAR) macrophages using a viral vector [8]. This method is complicated, expensive, and associated with virus-related safety concerns.

Embodiments of the subject invention are different from the methods discussed in the previous paragraph in that double stranded DNA (which can be referred to herein as simply “DNA” for simplification) can be used in the cytoplasm to promote M1 polarization. DNA in the cytoplasm can activate the cyclic GMP-AMP synthase (cGAS)-stimulator of interferon genes (STING)-interferon regulatory factor 3 (IRF3) pathway in a sequence-independent fashion, consequently polarizing the macrophage to M1 phenotype [12,13]. While DNA can be delivered into the cytoplasm of macrophages by using traditional nanometer-sized complexes (termed as nano-complexes) of DNA and supporting materials such as chitosan, polyethylenimine (PEI), calcium phosphate, and lipids, DNA in the nano-complexes can be degraded quickly by cytosolic exonucleases such as three-prime repair exonuclease 1 (TREX1) due to the small sizes of the nano-complexes [14,15,16,17]. Therefore, it is unlikely for these DNA-delivery methods to achieve sustained presence of the DNA in the cytoplasm and the resultant prolonged M1 polarization of the macrophages in an M2-promoting environment.

Embodiments of the subject invention can use highly engineered microparticles, as illustrated in FIGS. 1 and 2. Such a microparticle has a shape of a disk (e.g., a circular disk) and can have a diameter in a range of, for example, from 2 micrometers (μm) to 10 μm (e.g., 5 μm or about 5 μm) and can have a thickness in a range of, for example 0.1 μm to 3 μm (e.g., 1 μm or about 1 μm). The microparticle can include two components stacked together. One is the DNA/polycation multilayer, and the other is made of PNIPAM, which is a biocompatible polymer that is soluble in water at a temperature below 32° C. and insoluble at a temperature above 32° C. [18,19]. Microparticles with a similar structure have been produced with a similar method developed by the inventors of the subject invention [20]. The microparticles can be fed to cultured macrophages at a temperature of, for example, 37° C. and can be phagocytosed by the macrophages into their phagosomes [21-23]. The macrophages can then be exposed to a cold shock by replacing the heated (e.g., greater than 30° C., such as 37° C. or about 37° C.) medium with a low temperature (e.g., less than 10° C., such as 0° C. or about 0° C.) solution (e.g., phosphate-buffered saline (PBS)) for a predetermined amount of time (e.g., 2 minutes or about 2 minutes).

During the cold shock, the following events can happen sequentially: (1) dissolution of the PNIPAM layer in the phagosome; (2) a rapid increase of osmotic pressure in the phagosome; (3) burst of the phagosome induced by the high osmotic pressure; and (4) release of both the PNIPAM solution and the DNA/polycation multilayer into the cytoplasm. For an envisioned use of the macrophages for treating solid tumors, they can be infused back into the blood circulation of the patients and can accumulate in the solid tumors. At the human body temperature (37° C. or about 37° C.), the dissolved PNIPAM solution in the cytoplasm can condense into micrometer-sized droplets without exerting significant toxicity to the host macrophages. Given that a polycation-polyanion complex in water can be a liquid coacervate in the presence of salts, the DNA/polycation multilayer in the cytoplasm can assume such a state. This state of matter resembles the biomolecular condensate, which can be naturally formed by the negatively charged DNA and positively charged cGAS through liquid-liquid phase separation. Due to the natural affinity of cGAS between DNA, cGAS can enter the (PC/DNA) n multilayer. Because the natural DNA-cGAS biomolecular condensate can exclude TREX1 exonuclease, the (PC/DNA) n multilayer can also have this ability due to the high similarity between the DNA/polycation multilayer and the DNA-cGAS biomolecular condensate. Importantly, the significantly larger size of the multilayer compared to the nano-complexes can allow for much better protection of the DNA from the cytosolic TREX1 than the nano-complexes. Consequently, the DNA/polycation multilayer can allow a prolonged presence in the cytoplasm of the host macrophages and a continuous stimulation of the macrophages towards the M1 phenotype. DNA in a DNA-polycation multilayer in the cytoplasm of a macrophage can polarize the macrophage to M1 phenotype in an M2-promoting environment for an extended period.

Compared to related art systems and methods, embodiments of the subject invention have at least the following advantages. First, the DNA within the DNA/polycation multilayer can be shielded from enzymatic degradation in both phagosomes and the cytoplasm. Consequently, it can exert a prolonged effect on the cells, even in environments that inhibit M1 polarization. Second, the systems and methods of embodiments of the subject invention are versatile and applicable to a wide range of DNA, including double-stranded DNA of various sequences and origins and single-stranded DNA. Third, systems and methods of embodiments of the subject invention do not require genetic modification of the cells.

Solid-tumor-based cancer is a major cause of death in the United States. There is no effective therapy for treating metastatic solid tumors. AMT holds tremendous potential to become a viable therapy for this disease, but it is facing hurdles. Embodiments of the subject invention can overcome a major hurdle by being used either alone or in combination with other techniques (e.g., genetic modification) to render the macrophages with desirable therapeutic capabilities.

When ranges are used herein, combinations and subcombinations of ranges (including any value or subrange contained therein) are intended to be explicitly included. When the term “about” is used herein, in conjunction with a numerical value, it is understood that the value can be in a range of 95% of the value to 105% of the value, i.e. the value can be +/−5% of the stated value. For example, “about 1 kg” means from 0.95 kg to 1.05 kg.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

### Example 1

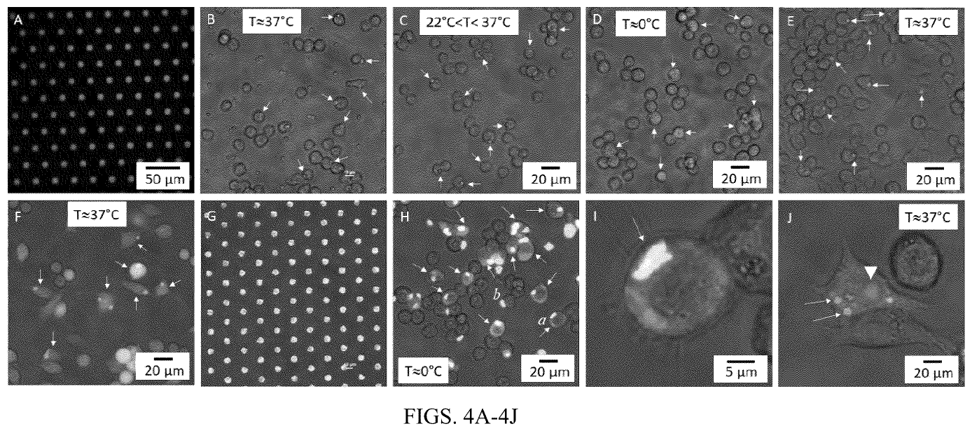

In embodiments of the subject invention, the PNIPAM layer of the microparticle must cause the phagosome to burst. To demonstrate its feasibility, microparticles composed of a single layer of PNIPAM were fabricated. To allow imaging of the microparticles, 100 nanometer (nm)-diameter red-fluorescent sulfate-modified polystyrene nanoparticles (NPs) were loaded in the microparticles. Briefly, the nanoparticles were dispersed in an aqueous solution of PNIPAM and the solution was spin-coated on a polydimethylsiloxane (PDMS) stamp bearing an array of 10 μm-diameter micropillars to form a dry PNIPAM film containing the nanoparticles on the stamp. The film on the top of the micropillars was printed on a glass coverslip as an array of disk-shaped microparticles denoted as PNIPAM (NPs) microparticles (see FIG. 4A) (see also [27]). The PNIPAM microparticles were stable in an aqueous solution at 37° C. and became dissolved in a 22° C. environment. RAW264.7 macrophages were added to the microparticles and incubated for 2 hours. The cells were then immediately imaged at 22° C.

Initially, as shown in FIG. 4B, microparticles that were not colocalized with any macrophage were visible and largely retained the arrayed pattern, and some microparticles were dislocated from their original locations and colocalized with macrophages (designated by arrows). All microparticles in FIG. 4B were around 5 μm in width, probably because the PNIPAM microparticles were hydrated and the hydrated microparticles shrank in the lateral dimensions to decrease their surface area [28]. After about 10 minutes, all microparticles that were not colocalized with any macrophage disappeared and most of the microparticles colocalized with macrophages remained. FIG. 4C shows 10 remaining microparticles (designated by arrows). The non-colocalized microparticles apparently dissolved as the temperature of the medium decreased and the remaining microparticles were absolutely inside the phagosomes of the macrophages. Otherwise, they would have been dissolved in either cytosol or medium.

### Example 2

In another experiment, the macrophages were incubated with the microparticles for 2 hours and the macrophages were immediately treated with the cold shock (0° C. for 2 minutes). Among all macrophages that contained red fluorescence, about 90% had diffuse red fluorescence, each of which covered a much larger area than the area covered by microparticles in the phagosomes before the cold shock. FIG. 4D shows 9 such macrophages (designated by arrows). The significant expansion of the red fluorescence indicates that the phagosomes burst, and the NPs spread into the cytoplasm. It is believed that the PNIPAM in the phagosomes became soluble in water at 0° C. and consequently created a high osmotic pressure inside the phagosomes. The pressure led to influx of water into the phagosomes, causing them to swell and eventually burst. As a result, the dissolved PNIPAM flew out of the ruptured phagosomes and entered the cytoplasm, with the nanoparticles being dispersed in PNIPAM solution.

The macrophages after the cold shock were further incubated in 37° C. medium. After 24 hours, no macrophage that contained the diffuse red fluorescence as in FIG. 4D was observed. Instead, for almost all macrophages that contained red fluorescence, the red-fluorescent objects in the macrophages appeared as circular droplets with a lateral size of about 4 μm or smaller. Moreover, macrophages that contained the red fluorescence typically contained one droplet per cell. FIG. 4E shows 10 droplets in 10 macrophages (designated by arrows). It is believed that the dissolved PNIPAM in the cytoplasm of the macrophages formed hydrated PNIPAM droplets during the 24-hour 37° C. culture period. Because the fluorescent nanoparticles were water-soluble, they were probably trapped in the PNIPAM solution in FIG. 4D and in the PNIPAM droplets in FIG. 4E. Because the fluorescent nanoparticles in a macrophage covered a much larger area at 0° C. than the microparticle at 37° C., the phagosomes were definitely burst at 0° C. Moreover, the PNIPAM droplets in FIG. 4E were unlikely covered by the original phagosomal membrane. In addition, it was found that about 70% of the cells that initially contained microparticles were alive after being cultured for 24 hours as assessed with calcein AM and trypan blue assays. FIG. 4F shows typical macrophages that contained PNIPAM (NPs) droplets after the cold shock and 24-hour culture and being stained by both calcein AM and trypan blue. Note that 7 cells in FIG. 4F contained one or more PNIPAM (NPs) droplets (designated by arrows). All of them were brightly stained by calcein AM and none was stained by trypan blue, indicating that they were alive.

### Example 3

To further demonstrate the feasibility of embodiments of the subject invention, microparticles were fabricated by using poly(allylamine hydrochloride) (PAH) as the polycation and DNA extracted from salmon testes. The DNA was stained by YOYO-1, which is a fluorescence dye and binds to DNA reversibly [29]. The microparticle structure is denoted as (PAH/DNA(YOYO-1))₂PNIPAM (NPs). FIG. 4G shows an array of such microparticles on a glass coverslip. Macrophages were added to the microparticles and incubated for 18 hours and then treated with the cold shock (0° C. for 2 minutes). Approximately 90% of the macrophages that contained both red and green fluorescence were found to contain micrometer-sized green-fluorescent objects stained by YOYO-1 and diffuse red fluorescence, indicating that PNIPAM (NPs) had spread in the cytoplasm and the green-fluorescent objects were (PAH/DNA(YOYO-1))₂ multilayers. FIG. 4H shows 12 such macrophages (designated by arrows). One of them (marked by an “a”) is magnified in FIG. 4I, which clearly shows diffuse red fluorescence in the cytoplasm and a green (PAH/DNA(YOYO-1))₂ multilayer (designated by an arrow). It is noted that some micrometer-sized YOYO-1-stained DNA did not colocalize with any macrophage or were apparently attached to the surface of some macrophages such as the one marked by a “b” in FIG. 4H. They were likely the (PAH/DNA(YOYO-1))₂ part of the microparticles that were not phagocytosed by the macrophages.

The macrophages in FIG. 4H were further cultured at 37° C. After 24 hours, the micrometer-sized red-fluorescent droplets were seen in some macrophages. However, few of the macrophages contained micrometer-sized green-fluorescent objects. FIG. 4J shows a macrophage that contained two red-fluorescent droplets (designated by arrows) and did not contain a bright green-fluorescent object. While the DNA in the (PAH/DNA(YOYO-1))₂ multilayers could have been enzymatically degraded in the cytoplasm, it is also possible that the YOYO-1 left the DNA in the multilayer and stained nucleic acid in the cytoplasm and DNA in the nuclei of the macrophage. Indeed, diffuse green fluorescence was seen in the cytoplasm. In addition, the nucleolus of the macrophage was stained by YOYO-1 (designated by a white triangle/arrowhead). A similar result was observed when YOYO-1-stained DNA was delivered into the cytoplasm [30], suggesting that the (PAH/DNA(YOYO-1))₂ multilayer was delivered into the cytoplasm of the macrophage. Taken together, Examples 1-3 demonstrate that the systems and methods of embodiments of the subject invention successfully allow delivering of the polycation-DNA multilayers into the cytoplasm of macrophages without significantly reducing viability of the macrophages.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein (including in the “References” section, if present) are incorporated by reference in their entirety, including all figures and tables, to the extent they are not inconsistent with the explicit teachings of this specification.

### REFERENCES

[1] H. Dillekås, M. S. Rogers, O. Straume, Are 90% of deaths from cancer caused by metastases?, Cancer Med. 8 (2019)5574-5576. https://doi.org/10.1002/cam4.2474.
[2] Engineering strategies to overcome the current roadblocks in CAR T cell therapy|Nature Reviews Clinical Oncology, (n.d.). https://www.nature.com/articles/s41571-019-0297-y (accessed Jan. 12, 2022).
[3] W. A. Lim, C. H. June, The Principles of Engineering Immune Cells to Treat Cancer, Cell. 168 (2017)724-740. https://doi.org/10.1016/j.cell.2017.01.016.
[4] S. G, G. G, M. A, A. P, Tumor-associated macrophages (TAM) as major players of the cancer-related inflammation, Journal of Leukocyte Biology. 86 (2009). https://doi.org/10.1189/jlb.0609385.
[5] Y. Lin, J. Xu, H. Lan, Tumor-associated macrophages in tumor metastasis: biological roles and clinical therapeutic applications, Journal of Hematology & Oncology. 12 (2019) 76. https://doi.org/10.1186/s13045-019-0760-3.
[6] L. Bonapace, M.-M. Coissieux, J. Wyckoff, K. D. Mertz, Z. Varga, T. Junt, M. Bentires-Alj, Cessation of CCL2 inhibition accelerates breast cancer metastasis by promoting angiogenesis, Nature. 515 (2014)130-133. https://doi.org/10.1038/nature13862.
[7] S. Lee, S. Kivimae, A. Dolor, F. C. Szoka, Macrophage-based cell therapies: The long and winding road, J Control Release. 240 (2016)527-540. https://doi.org/10.1016/j.jconrel.2016.07.018.
[8] M. Klichinsky, M. Ruella, O. Shestova, X. M. Lu, A. Best, M. Zeeman, M. Schmierer, K. Gabrusiewicz, N. R. Anderson, N. E. Petty, K. D. Cummins, F. Shen, X. Shan, K. Veliz, K. Blouch, Y. Yashiro-Ohtani, S. S. Kenderian, M. Y. Kim, R. S. O'Connor, S. R. Wallace, M. S. Kozlowski, D. M. Marchione, M. Shestov, B. A. Garcia, C. H. June, S. Gill, Human chimeric antigen receptor macrophages for cancer immunotherapy, Nat Biotechnol. 38 (2020)947-953. https://doi.org/10.1038/s41587-020-0462-y.
[9] C. W. Shields, M. A. Evans, L. L.-W. Wang, N. Baugh, S. Iyer, D. Wu, Z. Zhao, A. Pusuluri, A. Ukidve, D. C. Pan, S. Mitragotri, Cellular backpacks for macrophage immunotherapy, Sci Adv. 6 (2020) eaaz6579. https://doi.org/10.1126/sciadv.aaz6579.
[10] C.-X. Li, Y. Zhang, X. Dong, L. Zhang, M.-D. Liu, B. Li, M.-K. Zhang, J. Feng, X.-Z. Zhang, Artificially Reprogrammed Macrophages as Tumor-Tropic Immunosuppression-Resistant Biologics to Realize Therapeutics Production and Immune Activation, Advanced Materials. 31 (2019)1807211. https://doi.org/10.1002/adma.201807211.
[11] Carisma Therapeutics Inc, A Phase 1, First in Human Study of Adenovirally Transduced Autologous Macrophages Engineered to Contain an Anti-HER2 Chimeric Antigen Receptor in Subjects With HER2 Overexpressing Solid Tumors, clinicaltrials.gov, 2022. https://clinicaltrials.gov/ct2/show/NCT04660929 (accessed Jan. 20, 2022).
[12] L. Yu, P. Liu, Cytosolic DNA sensing by cGAS: regulation, function, and human diseases, Sig Transduct Target Ther. 6 (2021)1-15. https://doi.org/10.1038/s41392-021-00554-y.
[13] D. J. Cao, G. G. Schiattarella, E. Villalobos, N. Jiang, H. I. May, T. Li, Z. J. Chen, T. G. Gillette, J. A. Hill, Cytosolic DNA Sensing Promotes Macrophage Transformation and Governs Myocardial Ischemic Injury, Circulation. 137 (2018) 2613-2634. https://doi.org/10.1161/CIRCULATIONAHA.117.031046.
[14] C. D. Thompson, M. R. Frazier-Jessen, R. Rawat, R. P. Nordan, R. T. Brown, Evaluation of methods for transient transfection of a murine macrophage cell line, RAW 264.7, Biotechniques. 27 (1999)824-826, 828-830, 832. https://doi.org/10.2144/99274rr05.
[15] X. Zhou, B. Liu, X. Yu, X. Zha, X. Zhang, Y. Chen, X. Wang, Y. Jin, Y. Wu, Y. Chen, Y. Shan, Y. Chen, J. Liu, W. Kong, J. Shen, Controlled release of PEI/DNA complexes from mannose-bearing chitosan microspheres as a potent delivery system to enhance immune response to HBV DNA vaccine, J Control Release. 121 (2007) 200-207. https://doi.org/10.1016/j.jconrel.2007.05.018.
[16] S. Pereira-Lopes, T. Celhar, G. Sans-Fons, M. Serra, A.-M. Fairhurst, J. Lloberas, A. Celada, The exonuclease Trex1 restrains macrophage proinflammatory activation, J Immunol. 191 (2013)6128-6135. https://doi.org/10.4049/jimmunol.1301603.
[17] S. R. Simpson, W. O. Hemphill, T. Hudson, F. W. Perrino, TREX1-Apex predator of cytosolic DNA metabolism, DNA Repair (Amst). 94 (2020) 102894. https://doi.org/10.1016/j.dnarep.2020.102894.
[18] H. G. Schild, Poly(N-isopropylacrylamide): experiment, theory and application, Progress in Polymer Science. 17 (1992) 163-249. https://doi.org/10.1016/0079-6700 (92)90023-R.
[19] Z. Guo, S. Li, C. Wang, J. Xu, B. Kirk, J. Wu, Z. Liu, W. Xue, Biocompatibility and cellular uptake mechanisms of poly(N-isopropylacrylamide) in different cells, Journal of Bioactive and Compatible Polymers. 32 (2017) 17-31. https://doi.org/10.1177/0883911516648969.
[20] J. Xia, Z. Wang, D. Huang, Y. Yan, Y. Li, J. Guan, Asymmetric Biodegradable Microdevices for Cell-Borne Drug Delivery, ACS Publications. (2015). https://doi.org/10.1021/acsami.5b00613.
[21] R. S. Flannagan, V. Jaumouillé, S. Grinstein, The cell biology of phagocytosis, Annu Rev Pathol. 7 (2012)61-98. https://doi.org/10.1146/annurev-pathol-011811-132445.
[22] G. J. Cannon, J. A. Swanson, The macrophage capacity for phagocytosis, J Cell Sci. 101 (Pt 4) (1992)907-913.
[23] Z. Wang, J. Xia, P. T. Hoang, L. Sun, S. Luo, Z. Cheng, Y. Ren, T. Liu, J. Guan, Fabrication of carbon nanotube-laden microdevices for Raman labeling of macrophages, Biomed. Phys. Eng. Express. (2017) 025012. https://doi.org/10.1088/2057-1976/aa6207.
[24] Q. Wang, J. B. Schlenoff, The Polyelectrolyte Complex/Coacervate Continuum, Macromolecules. 47 (2014)3108-3116. https://doi.org/10.1021/ma500500q.
[25] M. Du, Z. J. Chen, DNA-induced liquid phase condensation of cGAS activates innate immune signaling, Science. 361 (2018)704-709. https://doi.org/10.1126/science.aat1022.
[26] W. Zhou, L. Mohr, J. Maciejowski, P. J. Kranzusch, cGAS phase separation inhibits TREX1-mediated DNA degradation and enhances cytosolic DNA sensing, Mol Cell. 81 (2021)739-755.e7. https://doi.org/10.1016/j.molcel.2021.01.024.
[27] W. Cheng, S. Kim, S. Zivkovic, H. Chung, Y. Ren, J. Guan, Specific Labelling of Phagosome-Derived Vesicles in Macrophages with a Membrane Dye Delivered with Microfabricated Microparticles, Acta Biomaterialia. (2022). https://doi.org/10.1016/j.actbio.2022.01.028.
[28] R. Pelton, Poly(N-isopropylacrylamide) (PNIPAM) is never hydrophobic, J Colloid Interface Sci. 348 (2010)673-674. https://doi.org/10.1016/j.jcis.2010.05.034.
[29] K. Kucharska, M. Pilz, K. Bielec, T. Kalwarczyk, P. Kuźma, R. Hołyst, Two Intercalation Mechanisms of Oxazole Yellow Dimer (YOYO-1) into DNA, Molecules. 26 (2021) 3748. https://doi.org/10.3390/molecules26123748.
[30] J. Lv, H. Chang, Y. Wang, M. Wang, J. Xiao, Q. Zhang, Y. Cheng, Fluorination on polyethylenimine allows efficient 2D and 3D cell culture gene delivery, J. Mater. Chem. B. 3 (2014)642-650. https://doi.org/10.1039/C4TB01447B.
[31] M. Dash, F. Chiellini, R. M. Ottenbrite, E. Chiellini, Chitosan-A versatile semi-synthetic polymer in biomedical applications, Progress in Polymer Science. 36 (2011)981-1014. https://doi.org/10.1016/j.progpolymsci.2011.02.001.
[32] K. Tanaka, Y. Okahata, A DNA-Lipid Complex in Organic Media and Formation of an Aligned Film1, J. Am. Chem. Soc. 118 (1996) 10679-10683. https://doi.org/10.1021/ja9617855.
[33] X. Li, C. Shu, G. Yi, C. T. Chaton, C. L. Shelton, J. Diao, X. Zuo, C. C. Kao, A. B. Herr, P. Li, Cyclic GMP-AMP Synthase Is Activated by Double-Stranded DNA-Induced Oligomerization, Immunity. 39 (2013) 1019-1031. https://doi.org/10.1016/j.immuni.2013.10.019.
[34] P. Zhang, Y. Liu, J. Xia, Z. Wang, B. Kirkland, J. Guan, Top-down fabrication of polyelectrolyte-thermoplastic hybrid microparticles for unidirectional drug delivery to single cells, Adv Healthc Mater. 2 (2013) 540-545. https://doi.org/10.1002/adhm.201200200.
[35] P. Zhang, J. Guan, Fabrication of multilayered microparticles by integrating layer-by-layer assembly and microcontact printing, Small. 7 (2011) 2998-3004. https://doi.org/10.1002/smll.201101238.
[36] S. Das, A. Kumar, A. Mandal, K. Abhishek, S. Verma, A. Kumar, P. Das, Nucleic acid sensing activates the innate cytosolic surveillance pathway and promotes parasite survival in visceral leishmaniasis, Sci Rep. 9 (2019)9825. https://doi.org/10.1038/s41598-019-45800-0.
[37] S. P. C. Hsu, Y.-C. Chen, H.-C. Chiang, Y.-C. Huang, C.-C. Huang, H.-E. Wang, Y.-S. Wang, K.-H. Chi, Rapamycin and hydroxychloroquine combination alters macrophage polarization and sensitizes glioblastoma to immune checkpoint inhibitors, J Neurooncol. 146 (2020)417-426. https://doi.org/10.1007/s11060-019-03360-3.
[38] L. Liu, Y. Wang, X. Guo, J. Zhao, S. Zhou, A Biomimetic Polymer Magnetic Nanocarrier Polarizing Tumor-Associated Macrophages for Potentiating Immunotherapy, Small. 16 (2020)2003543. https://doi.org/10.1002/smll.202003543.
[39] H.-C. Lee, K. Chathuranga, J.-S. Lee, Intracellular sensing of viral genomes and viral evasion, Exp Mol Med. 51 (2019)1-13. https://doi.org/10.1038/s12276-019-0299-y.

What is claimed is:

1. A method for maintaining an immune cell in a solid tumor in a tumoricidal M1 phenotype, the method comprising:
a) incubating the immune cell with a microdisk comprising a layer of poly(N-isopropylacrylamide) (PNIPAM) and a multilayer of deoxyribonucleic acid (DNA) and a polycation (PC), the incubating being performed at a first temperature for a first period of time;
b) after step a), exposing the immune cell and the microdisk to a second temperature of less than 10° C. for a second period of time; and
c) after step b), culturing the immune cell at a third temperature for a third period of time, the third temperature being at least 30° C.

2. The method according to claim 1, wherein the first temperature is at least 30° C.

3. The method according to claim 1, wherein the second temperature is about 0° C.

4. The method according to claim 1, wherein the first temperature is about 37° C.

5. The method according to claim 1, wherein the third temperature is about 37° C.

6. The method according to claim 1, wherein step a) causes the microdisk to be phagocytosed by the immune cell.

7. The method according to claim 1, wherein step b) causes phagosomal rupture and release of the multilayer of DNA and PC into a cytoplasm of the immune cell.

8. The method according to claim 1, wherein step b) causes the PNIPAM to dissolve.

9. The method according to claim 1, wherein the first period of time is in a range of from 0.5 hours to 30 hours,
wherein the second period of time is in a range of from 0.5 minutes to 10 minutes, and
wherein the third period of time is in a range of from 10 hours to 48 hours.

10. The method according to claim 1, wherein the first period of time is about 2 hours.

11. The method according to claim 1, wherein the second period of time is about 2 minutes.

12. The method according to claim 1, wherein the third period of time is about 24 hours.

13. The method according to claim 1, wherein the microdisk has a thickness in a range of from 0.1 micrometers (μm) to 3 μm and a diameter in a range of from 2 μm to 10 μm.

14. The method according to claim 13, wherein the thickness of the microdisk is about 1 μm, and wherein the diameter of the microdisk is about 5 μm.

15. The method according to claim 1, further comprising, before step a):
i) depositing the multilayer of DNA and PC on a stamp comprising pillars;
ii) depositing the layer of PNIPAM on the multilayer of DNA and PC;
iii) printing the microdisk on a substrate by disposing the stamp comprising the layer of PNIPAM and the multilayer of DNA and PC on the substrate.

16. The method according to claim 15, further comprising, before step iii), coating the substrate with a polymer.

17. The method according to claim 16, where the polymer is polyvinyl alcohol (PVA).

18. The method according to claim 1, wherein the immune cell is a macrophage or a monocyte.

19. The method according to claim 18, wherein the immune cell is an adoptively transferred macrophage.

20. A method of treating a disease in a patient, the method comprising:
isolating immune cells from the patient;
modifying the immune cells ex vivo by applying the method according to claim 1 to the immune cells; and
re-infusing the modified immune cells back to the patient.
